(19) United States
(12) Patent Application Publication
(10) Pub. No.: US 2009/0230854 A1
(43) Pub. Date: Sep. 17, 2009

(54) Indeno indene-based compound, organic light emitting device comprising the same, and method of manufacturing the organic light emitting device

(76) Inventors: Myeong-suk Kim, Hwaseong-si (KR); Byoung-ki Choi, Hwaseong-si (KR); Yu-jin Kim, Suwon-si (KR); Dong-woo Shin, Seoul (KR); Tae-yong Noh, Seoul (KR); O-hyun Kwon, Seoul (KR); Tae-woo Lee, Seoul (KR)

Correspondence Address: ROBERT E. BUSHNELL & LAW FIRM, 2029 K STREET NW, SUITE 600, WASHINGTON, DC, 20006-1004, US

(21) Appl. No.: 12/382,355

(22) Filed: Mar. 13, 2009

(30) Foreign Application Priority Data
Mar. 14, 2008 (KR) 10-2008-0024060

(51) Int. Cl. H01J 1/62; C07C 13/465; C07C 211/54; B05D 5/06

(52) U.S. Cl. 313/504; 585/27; 564/426; 427/162

(57) ABSTRACT

Provided is an indeno indene-based compound represented by Formula 1:

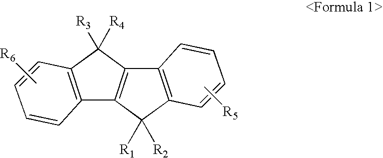

where R₁, R₂, R₃, R₄, R₅ and R₆ are the same as in the detailed description. An organic light emitting device including the indeno indene-based compound has low driving voltage, high efficiency, and high color purity.

### CROSS-REFERENCE TO RELATED PATENT APPLICATIONS AND CLAIM OF PRIORITY

This application claims the benefit of Korean Patent Application No. 10-2008-0024060, filed on Mar. 14, 2008, in the Korean Intellectual Property Office, the disclosure of which is incorporated herein in its entirety by reference.

### BACKGROUND OF THE INVENTION

1. Field of the Invention

The present invention relates to an indeno indene-based compound, an organic light emitting device (OLED) including the same, and a method of manufacturing the OLED, and more particularly, to an indeno indene-based compound having excellent electrical properties, thermal stability and photochemical stability, and being capable of improving driving voltage properties, efficiency, and color purity when used in an organic light emitting device, an organic light emitting device including the indeno indene-based compound, and a method of manufacturing the organic light emitting device.

2. Description of the Related Art

Light emitting devices are self-emission devices, and have wide viewing angles, excellent contrast properties, and quick response speeds. Light emitting devices can be categorized into inorganic light emitting devices including an emission layer formed of an inorganic compound and OLEDs including an emission layer formed of an organic compound. Compared to inorganic light emitting devices, OLEDs have excellent brightness, low driving voltages, and quick response speeds, and can produce full-color images. Therefore, research into OLEDs is being actively performed.

In general, an OLED has a structure of anode/organic emission layer/cathode. An OLED can have other structures, such as a structure of anode/hole injection layer/hole transport layer/emission layer/electron transport layer/electron injection layer/cathode, or a structure of anode/hole injection layer/hole transport layer/emission layer/hole blocking layer/electron transport layer/electron injection layer/cathode.

JP Patent Publication No. 1999-003782 discloses an anthracene substituted with two naphthyl groups, which can be used to form an emission layer or a hole injection layer. However, an OLED including such a compound does not have sufficient driving voltage, brightness, efficiency, and color purity properties. Thus, there is a need to develop an OLED having desirable low driving voltage, high brightness, high efficiency, and high color purity properties.

### SUMMARY OF THE INVENTION

The present invention provides a novel indeno indene-based compound.

An embodiment of the present invention provides an indeno indene-based compound capable of improving driving voltage properties, efficiency, and color purity when used in an organic light emitting device, an organic light emitting device including the indeno indene-based compound, and a method of manufacturing the organic light emitting device.

According to an aspect of the present invention, there is provided an indeno indene-based compound represented by Formula 1:

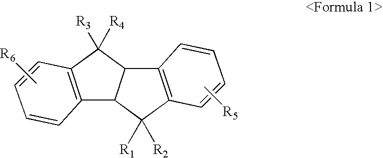

where R₁, R₂, R₃, R₄, R₅ and R₆ are each independently, hydrogen, a substituted or unsubstituted C₁-C₄₀ alkyl group, a substituted or unsubstituted C₁-C₄₀ alkoxy group, a substituted or unsubstituted C₆-C₅₀ aryl group, a substituted or unsubstituted C₂-C₅₀ heteroaryl group, a substituted or unsubstituted C₅-C₄₀ cycloalkyl group, a substituted or unsubstituted C₅-C₄₀ hetero cycloalkyl group, —N(Q₁)(Q₂) or —Si(Q₃)(Q₄)(Q₅) where Q₁, Q₂, Q₃, Q₄ and Q₅ are each independently, hydrogen, a substituted or unsubstituted C₆-C₅₀ aryl group, a substituted or unsubstituted C₂-C₅₀ heteroaryl group, a substituted or unsubstituted C₅-C₄₀ cycloalkyl group or a substituted or unsubstituted C₅-C₄₀ hetero cycloalkyl group.

In Formula 1, R₁, R₂, R₃, R₄, R₅ and R₆ may be each independently aryl groups having the following structures:

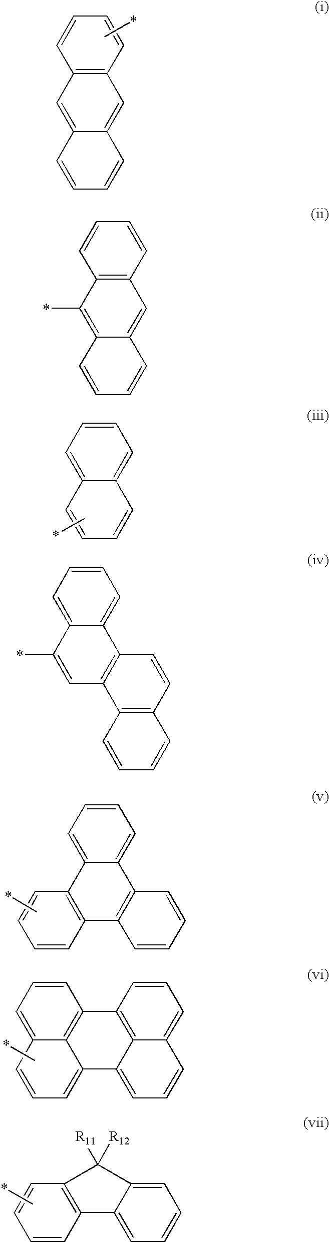

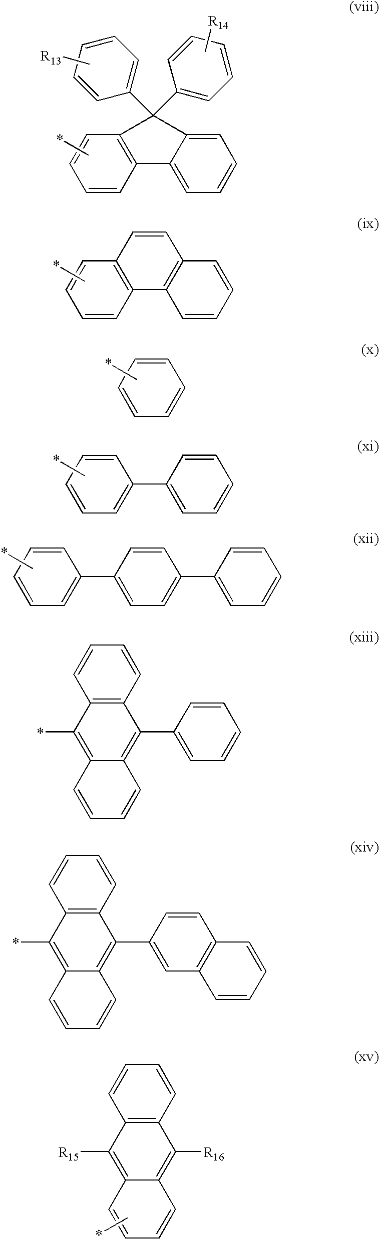

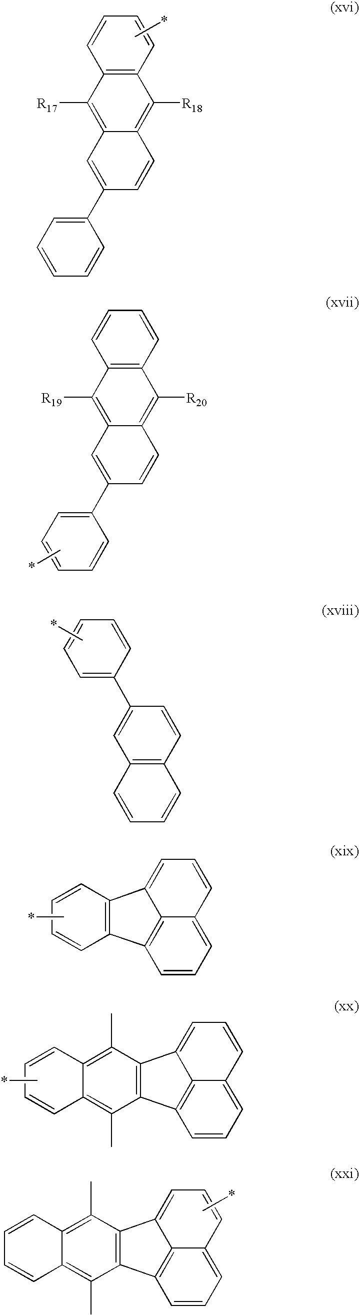

where R₁₁, R₁₂, R₁₃, R₁₄, R₁₅, R₁₆, R₁₇, R₁₈, R₁₉ and R₂₀ are each independently, hydrogen, a substituted or unsubstituted C₁-C₄₀ alkyl group, a substituted or unsubstituted C₆-C₅₀ aryl group, a substituted or unsubstituted C₂-C₅₀ heteroaryl group, a substituted or unsubstituted C₅-C₄₀ cycloalkyl group or a substituted or unsubstituted C₅-C₄₀ hetero cycloalkyl group; and

an asterisk (*) of each structure respectively represents a binding site.

According to another aspect of the present invention, there is provided an organic light emitting device including: a first electrode; a second electrode; and at least one organic layer between the first electrode and the second electrode, wherein the organic layer comprises the indeno indene-based compound according to an embodiment of the present invention.

According to another aspect of the present invention, there is provided a method of manufacturing an organic light emitting device, the method comprising: forming a first electrode on a substrate; forming an organic layer comprising the indeno indene-based compound on the first electrode; and forming a second electrode on the organic layer.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings in which like reference symbols indicate the same or similar components, wherein:

FIGS. 1A through 1C illustrate schematic stacked structures of organic light emitting devices according to embodiments of the present invention; and

FIG. 2 illustrates an absorption spectrum of a film formed of an indeno indene-based compound according to an embodiment of the present invention.

### DETAILED DESCRIPTION OF THE INVENTION

The present invention will now be described more fully with reference to the accompanying drawings, in which exemplary embodiments of the invention are shown.

An indeno indene-based compound according to the present invention is represented by Formula 1:

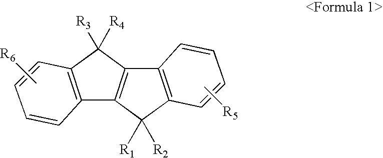

where R₁, R₂, R₃, R₄, R₅ and R₆ are each independently, hydrogen, a substituted or unsubstituted C₁-C₄₀ alkyl group, a substituted or unsubstituted C₁-C₄₀ alkoxy group, a substituted or unsubstituted C₆-C₅₀ aryl group, a substituted or unsubstituted C₂-C₅₀ heteroaryl group, a substituted or unsubstituted C₅-C₄₀ cycloalkyl group, a substituted or unsubstituted C₅-C₄₀ hetero cycloalkyl group, —N(Q₁)(Q₂) or —Si(Q₃)(Q₄)(Q₅) where Q₁, Q₂, Q₃, Q₄ and Q₅ are each independently, hydrogen, a substituted or unsubstituted C₆-C₅₀ aryl group, a substituted or unsubstituted C₂-C₅₀ heteroaryl group, a substituted or unsubstituted C₅-C₄₀ cycloalkyl group or a substituted or unsubstituted C₅-C₄₀ hetero cycloalkyl group.

An indeno indene moiety may contribute to an increase in thermal stability and photochemical stability, and thus a compound including the indeno indene can have excellent properties, such as a long lifetime. Meanwhile, the indeno indene-based compound according to the present invention has high solubility and high film proccesibility due to inclusion of R₁ through R₆ bonded to the indeno indene moiety of Formula 1. Therefore, the indeno indene-based compound can be used to form an organic layer of an organic light emitting device. For example, the organic layer can be a hole injection layer, a hole transport layer, an emission layer, an electron injection layer or an electron transport layer. Specifically, the indeno indene moiety of Formula 1 can be used as a light emitting material of an organic light emitting device, and thus, can act as a host or dopant of an emission layer.

Specifically, R₁, R₂, R₃, R₄, R₅ and R₆ are each independently, hydrogen, a C₁-C₄₀ alkyl group, a C₁-C₄₀ alkoxy group, a phenyl group, a biphenyl group, a pentalenyl group, an indenyl group, a naphthyl group, a biphenylenyl group, an anthracenyl group, an azurenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolinyl group, a piperidinyl group, a piperazinyl group, a morpholinyl group, a di(C₆-C₅₀ aryl)amino group, a tri(C₆-C₅₀aryl)silyl group, or derivatives thereof.

When R₁, R₂, R₃, R₄, R₅ and R₆ are aryl groups, R₁, R₂, R₃, R₄, R₅ and R₆ may each independently have any one aryl structure selected from the following structures:

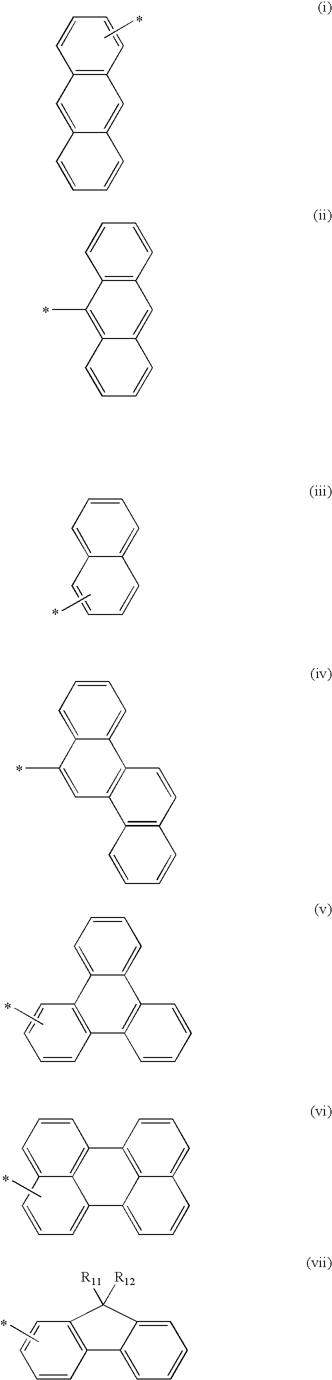

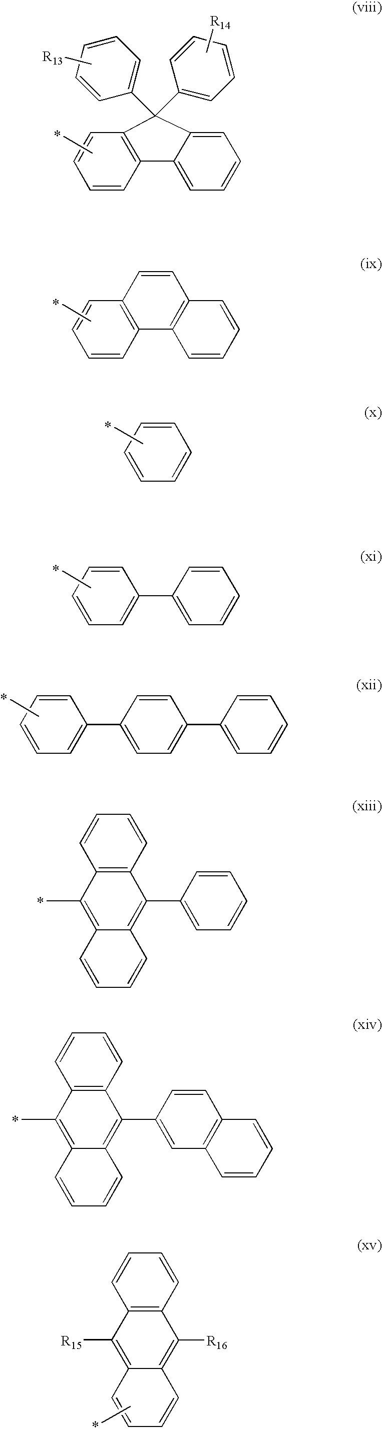

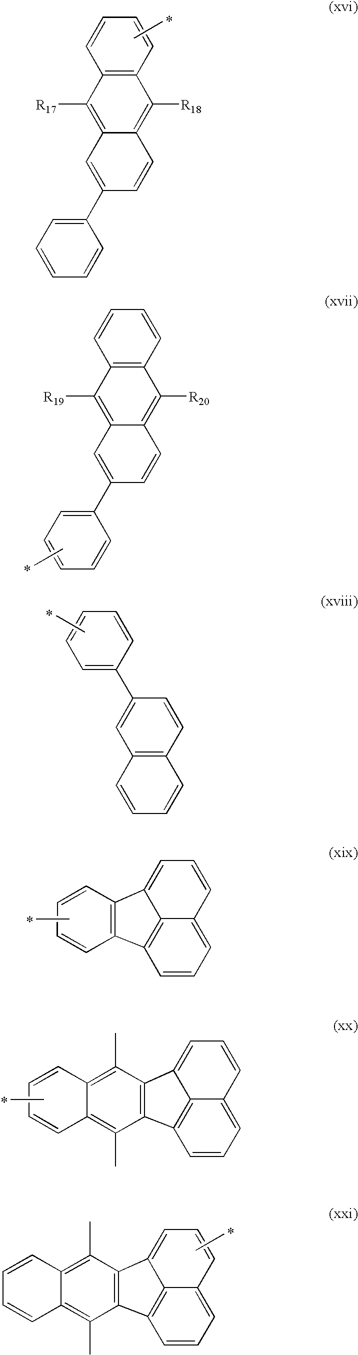

where R₁₁, R₁₂, R₁₃, R₁₄, R₁₅, R₁₆, R₁₇, R₁₈, R₁₉ and R₂₀ are each independently, hydrogen, a substituted or unsubstituted C₁-C₄₀ alkyl group, a substituted or unsubstituted C₆-C₅₀ aryl group, a substituted or unsubstituted C₂-C₅₀ heteroaryl group, a substituted or unsubstituted C₅-C₄₀ cycloalkyl group or a substituted or unsubstituted C₅-C₄₀ hetero cycloalkyl group; and

an asterisk (*) of each structure respectively represents a binding site.

According to embodiments of the present invention, R₁, R₂, R₃, R₄, R₅ and R₆ in Formula 1 may be each independently selected from the group consisting of hydrogen, a methyl group, and functional groups represented by Formulae iii, x, xviii and xxii:

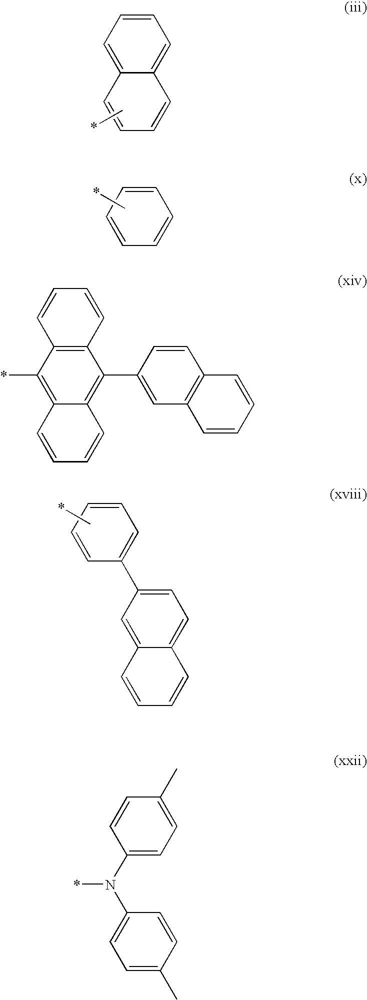

where an asterisk (*) indicates a binding site.

According to another embodiment of the present invention, at least one of R₁ and R₂ and at least one of R₃ and R₄ are each independently selected from the group consisting of a methyl group and functional groups represented by Formulae iii, x, xviii and xxii.

The indeno indene-based compound according to embodiments of the present invention provides good major light emission characteristics and thermal stability due to inclusion of the aryl group.

In addition, due to inclusion of R₁, R₂, R₃, R₄, R₅ and R₆, the light emission wavelength can be adjusted, and thermal stability, film properties, and optical stability can be improved. Specifically, due to inclusion of R₁, R₂, R₃, R₄, R₅ and R₆, an amorphous film having excellent properties at room temperature can be manufactured.

Examples of an unsubstituted alkyl group used in formulae of the present invention may be methyl, ethyl, propyl, isobutyl, sec-butyl, tert-butyl, pentyl, iso-amyl, or hexyl, and at least one hydrogen atom of the unsubstituted alkyl group may be substituted with a substituent to be a substituted alkyl group used in formulae of the present invention.

An unsubstituted cycloalkyl group used in formulae of the present invention may be an alkyl group having a ring system. A heterocycloalkyl group may be obtained by substituting at least one carbon of the cycloalkyl group with at least one element selected from N, O, S, and P. Examples of the unsubstituted cycloalkyl group include a cyclohexyl group or a cyclopentyl group, and at least one hydrogen atom of the unsubstituted cycloalkyl group may be substituted with a substituent.

Examples of an unsubstituted alkoxy group used in formulae of the present invention may be methoxy, ethoxy, phenyloxy, cyclohexyloxy, naphthyloxy, isopropyloxy, or diphenyloxy. Examples of a substituted alkoxy group used in formulae of the present invention may be compounds in which at least one hydrogen atom of the unsubstituted alkoxy group is substituted with a substituent.

An aryl group used in formulae of the present invention is a carbocyclic aromatic system having at least one aromatic group, and, when two or more aromatic groups are included, at least one ring may be pendent or fused with another aromatic group. Examples of an unsubstituted aryl group include a phenyl group, an ethylphenyl group, an ethylbiphenyl group, an o-, m- and p-fluorophenyl group, a dichlorophenyl group, a dicyanophenyl group, a trifluoromethoxyphenyl group, an o-, m-, and p-tolyl group, an o-, m- and p-kumenyl group, a mesityl group, a phenoxyphenyl group, a (α,α-dimethylbenzene)phenyl group, a (N,N′-dimethyl)aminophenyl group, a pentalenyl group, an indenyl group, a naphthyl group, a methylnaphthyl group, an anthracenyl group, an azurenyl group, a heptalenyl group, an acenaphthylenyl group, a phenalenyl group, a fluorenyl group, an anthraquinolyl group, a methylanthryl group, a penanthryl group, a triphenylene group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a pyranthrenyl group, an ovalenyl group, and a carbazolyl group. At least one hydrogen atom of the unsubstituted aryl group may be substituted with a substituent.

An unsubstituted heteroaryl group used in formulae of the present invention may be obtained by substituting at least one carbon of the aryl group with at least one element selected from N, O, P and S. In the heteroaryl group, at least one hydrogen atoms can be substituted with a substituent to be a substituted heteroaryl group. Examples of the unsubstituted heteroaryl group include a pyrazolyl group, an imidazolyl group, an oxazolyl group, a thiazolyl group, a triazolyl group, a tetrazolyl group, an oxadiazolyl group, a pyridinyl group, a pyridazinyl group, a pyrimidinyl group, a triazinyl group, a carbazolyl group, and an indolyl group.

An unsubstituted aryl amino group used in formulae of the present invention may be represented by —Ar—N(Q₆)(Q₇) where Q₆ and Q₇ are each independently hydrogen, a substituted or unsubstituted C₁-C₂₀ alkyl group, a substituted or unsubstituted C₆-C₃₀ aryl group, a substituted or unsubstituted C₂-C₃₀ heteroaryl group, a substituted or unsubstituted C₅-C₂₀ cycloalkyl group or a substituted or unsubstituted C₅-C₃₀ heterocycloalkyl group. Examples of the unsubstituted aryl amino group include a diphenylamino group.

An unsubstituted arylsilyl group used in formulae of the present invention is represented by —Ar—Si(Q₈)(Q₉)(Q₁₀) where Q₈, Q₉, and Q₁₀ are each independently hydrogen, a substituted or unsubstituted C₁-C₂₀ alkyl group, a substituted or unsubstituted C₆-C₃₀ aryl group, a substituted or unsubstituted C₂-C₃₀ heteroaryl group, a substituted or unsubstituted C₅-C₂₀ cycloalkyl group or a substituted or unsubstituted C₅-C₃₀ heterocycloalkyl group.

The term “substituted” used to define substituents used in formulae of the present invention refers to substituting a functional group with any substituent. Examples of the substituent may include at least one substituent selected from the group consisting of —F; —Cl; —Br; —CN; —NO₂; —OH; a C₁-C₅₀ alkyl group that is unsubstituted or substituted with —F, —Cl, —Br, —CN, —NO₂ or —OH; a C₁-C₅₀ alkoxy group that is unsubstituted or substituted with —F, —Cl, —Br, —CN, —NO₂ or —OH; a C₆-C₅₀ aryl group that is unsubstituted or substituted with a C₁-C₅₀ alkyl group, a C₁-C₅₀ alkoxy group, —F, —Cl, —Br, —CN, —NO₂ or —OH; a C₂-C₅₀ heteroaryl group that is unsubstituted or substituted with a C₁-C₅₀ alkyl group, a C₁-C₅₀ alkoxy group, —F, —Cl, —Br, —CN, —NO₂ or —OH; a C₅-C₅₀ cycloalkyl group that is unsubstituted or substituted with a C₁-C₅₀ alkyl group, a C₁-C₅₀ alkoxy group, —F, —Cl, —Br, —CN, —NO₂ or —OH; a C₅-C₅₀ heterocycloalkyl group that is unsubstituted or substituted with a C₁-C₅₀ alkyl group, a C₁-C₅₀ alkoxy group, —F, —Cl, —Br, —CN, —NO₂ or —OH; or a group represented by —N(Q₁₁)(Q₁₂) where Q₁₁ and Q₁₂ are each independently hydrogen, a C₁-C₅₀ alkyl group, or a C₆-C₅₀ aryl group substituted with a C₁-C₅₀ alkyl group.

In the present specification, the term “derivative” refers to a group obtained by substituting at least one atom of the groups described above with at least one substituent described above. For example, it would be obvious to one of ordinary skill in the art that the derivative includes a tolyl group, a methoxy-naphthyl group, or the like. Specifically, the substituent may be methyl, methoxy, a phenyl group, a tolyl group, a naphthyl group, a pyrenyl group, a phenanthrenyl group, a fluorenyl group, an imidazolinyl group, an indolyl group, a quinolinyl group, a diphenylamino group, a 2,3-di-p-toylaminophenyl group, or a triphenylsilyl group.

Specifically, the indeno indene-based compound represented by Formula 1 may have any one of the structures represented by Formulae 2 through 7 below, but is not limited thereto.

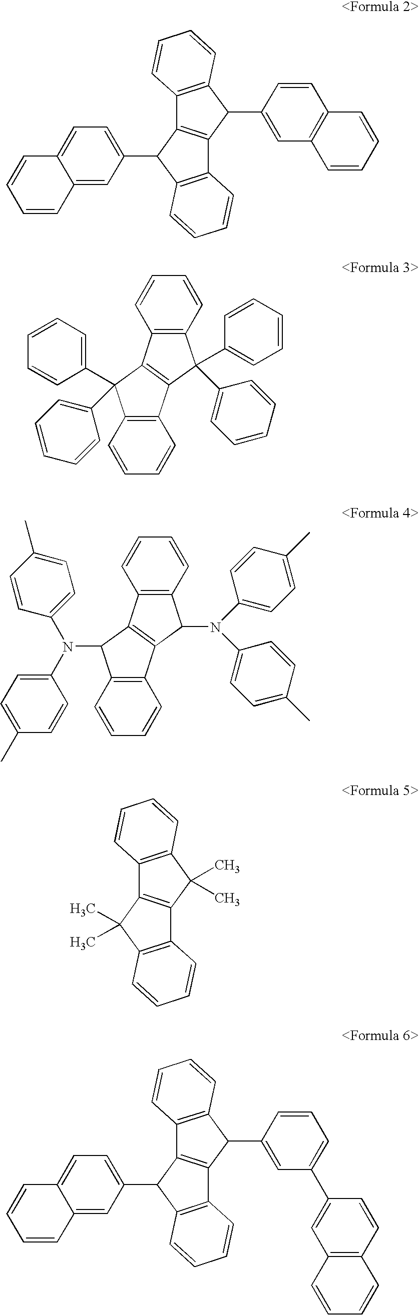

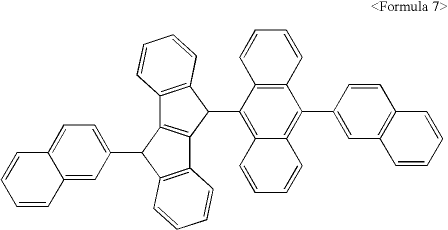

The indeno indene-based compound according to embodiments of the present invention can be synthesized by using a known synthesis method. The exemplary synthetic pathway of the indeno indene-based compound is shown in detail in Reaction Schemes of Synthesis Example 1 which will be described later.

The indeno indene-based compound according to embodiments of the present invention can be used in an organic light emitting device. An organic light emitting device according to an embodiment of the present invention includes a first electrode; a second electrode; and at least one organic layer interposed between the first electrode and the second electrode, wherein the organic layer includes the indeno indene-based compound represented by Formula 1 according to an embodiment of the present invention. Specifically, in the organic light emitting device according to the present invention, the compound represented by Formula 1 may be any one compound selected from the compounds represented by Formulae 2 through 7.

The organic layer including the indeno indene-based compound can be a hole injection layer, a hole transport layer, an electron blocking layer, an emission layer, a hole blocking layer, an electron transport layer or an electron injection layer. According to embodiments of the present invention, since the indeno indene-based compound represented by Formula 1 can efficiently function as a hole injection layer forming material, a hole transport layer forming material, or a host or dopant material of an emission layer, the organic layer can be a hole injection layer, a hole transport layer, or an emission layer. The emission layer may further include, in addition to the indeno indene-based compound according to the present invention, other light emitting material.

The organic layer may be formed by wet spinning or thermal transferring, such as spin coating, inkjet printing, or spray printing. However, the organic layer can also be formed using other methods.

The organic light emitting device according to embodiments of the present invention may have various structures. That is, the organic light emitting device according to the present invention may further include, between the first electrode and the second electrode, at least one layer selected from the group consisting of a hole injection layer, a hole transport layer, a hole blocking layer, an electron blocking layer, an electron transport layer and an electron injection layer.

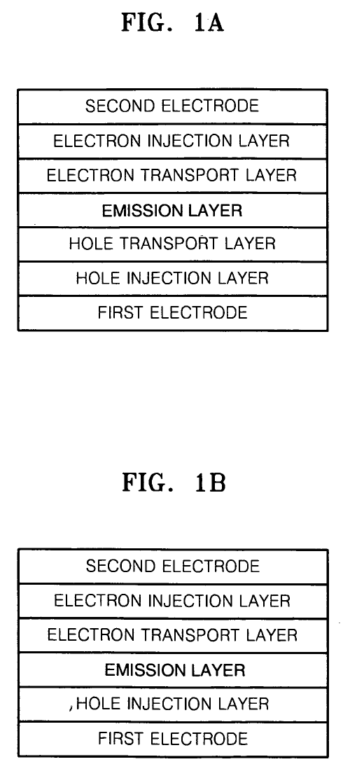

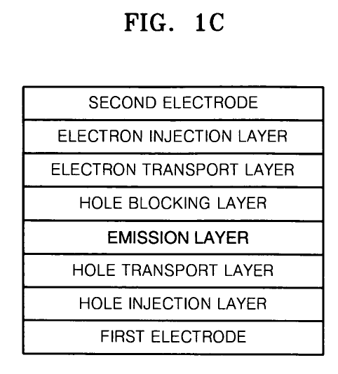

FIGS. 1A through 1C illustrate schematic stacked structures of organic light emitting devices according to embodiments of the present invention. The organic light emitting device illustrated in FIG. 1A has a structure of first electrode/hole injection layer/hole transport layer/emission layer/electron transport layer/electron injection layer/second electrode. The organic light emitting device illustrated in FIG. 1B has a structure of first electrode/hole injection layer/hole transport layer/emission layer/electron transport layer/electron injection layer/second electrode. The organic light emitting device illustrated in FIG. 1C has a structure of first electrode/hole injection layer/hole transport layer/emission layer/hole blocking layer/electron transport layer/electron injection layer/second electrode. At least one layer selected from the group consisting of the electron injection layer, the electron transport layer, the hole blocking layer, the emission layer, the hole injection layer and the hole transport layer may include the indeno indene-based compound according to the present invention.

The emission layer of an organic light emitting device according to an embodiment of the present invention may further include a red, green, blue, or white phosphorescent or fluorescent dopant. Among those dopants, the red, green, blue, or white fluorescent dopant may be an organometallic compound including at least one element selected from the group consisting of Ir, Pt, Os, Ti, Zr, Hf, Eu, Tb and Tm.

A method of manufacturing an organic light emitting device according to an embodiment of the present invention may include: forming a first electrode on a substrate; forming an organic layer including the indeno indene-based compound described above on the first electrode; and forming a second electrode on the organic layer. The organic layer including the indeno indene-based compound can be a hole injection layer, a hole transport layer, an electron blocking layer, an emission layer, a hole blocking layer, an electron transport layer or an electron injection layer, and specifically, a hole injection layer, a hole transport layer or an emission layer.

The organic layer may be formed using various known methods. For example, the organic layer can be formed using a known deposition method, a spin coating method, an inkjet printing method, a casting method, a Langmuir Blodgett (LB) deposition method, or a microcontact printing method. However, the organic layer can also be formed using other methods.

The method of manufacturing an organic light emitting device may further include at least one process selected from the group consisting of a hole injection layer forming process, a hole transport layer forming process, an electron blocking layer forming process, a hole blocking layer forming process, an electron transport layer forming process, and an electron injection layer forming process, according to a stack structure of the desired organic light emitting device,

A method of manufacturing an organic light emitting device according to an embodiment of the present invention will now be described in detail with reference to the organic light emitting device of FIG. 1C.

First, a first electrode forming material having a high work function is applied to a substrate by deposition or sputtering, thereby forming a first electrode. The first electrode may function as an anode. The substrate may be any substrate that is used in conventional organic light emitting devices. For example, the substrate may be a glass substrate, or a transparent plastic substrate having high mechanical strength, high thermal stability, high transparency, a planar surface, convenience for handling, and excellent waterproof properties. The first electrode forming material may be a transparent and highly conductive material, such as indium tin oxide (ITO), indium zinc oxide (IZO), tin oxide (SnO₂), or zinc oxide (ZnO).

Then, a hole injection layer (HIL) can be formed on the first electrode by using a vacuum deposition method, a spin coating method, a casting method, or an LB deposition method.

When the HIL is formed using a vacuum deposition method, the deposition conditions may differ according to the type of a HIL forming material and the structure and thermal properties of the desired HIL. For example, the deposition temperature may be in a range of 100 to 500° C., the degree of vacuum may be in a range of 10⁻⁸ to 10⁻³ torr, and the deposition rate may be in a range of 0.01 to 100 Å/sec.

When the HIL is formed using a spin coating method, the coating condition may differ according to the type of a HIL forming material and the structure and thermal properties of the desired HIL. For example, the coating rate may be in a range of about 2000 rpm to 5000 rpm, and a heat treatment temperature at which a solvent used in the coating process is removed may be in a range of about 80° C. to 200° C.

The HIL forming material may be the indeno indene-based compound represented by Formula 1. In addition, the HIL forming material may be any known hole injecting material, such as: a copper phthalocyanine compound disclosed in U.S. Pat. No. 4,356,429 which is incorporated herein by reference; a star-burst type amine derivative, such as TCTA, m-MTDATA, or m-MTDAPB, disclosed in Advanced Material, 6, p.677 (1994) which is incorporated herein by reference; or a soluble and conductive polymer, such as polyaniline/dodecylbenzenesulfonic acid (Pani/DBSA), poly(3,4-ethylenedioxythiophene)/poly(4-styrenesulfonate (PEDOT/PSS)), polyaniline/camphor sulfonicacid (Pani/CSA), or polyaniline/poly(4-styrenesulfonate) (PANI/PSS).

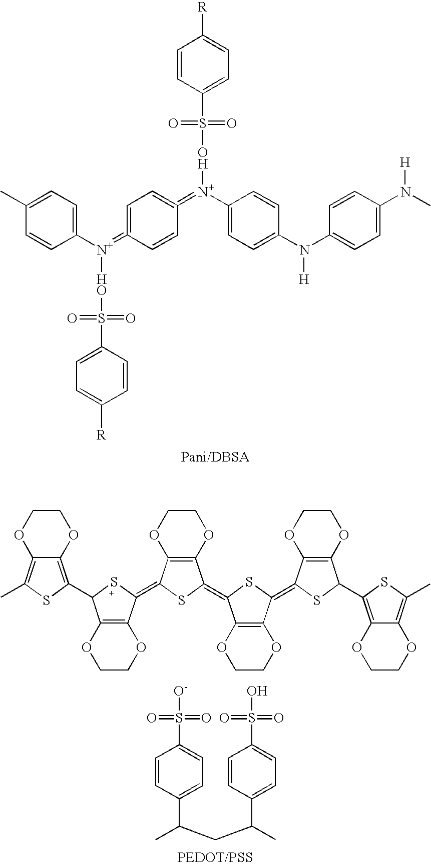

The thickness of the HIL may be in a range of about 100 Å to 10,000 Å, specifically 100 Å to 1,000 Å. If the thickness of the HIL is less than 100 Å, hole injecting properties may be degraded. On the other hand, if the thickness of the HIL is greater than 10,000 Å, the driving voltage of the organic light emitting device may be increased.

Then, a hole transport layer (HTL) can be formed on the HIL by using a vacuum deposition method, a spin coating method, a casting method, or an LB deposition method. When the HTL is formed by using a vacuum deposition method or a spin coating method, the deposition and coating conditions may differ according to the type of a HTL forming material, and can be very similar to the deposition and coating conditions used to form the HIL.

The HTL forming material may be the indeno indene-based compound represented by Formula 1. The HTL forming material may also be any known hole transporting material. In this regard, the HTL forming material may be a carbazole derivative, such as N-phenylcarbazole or polyvinylcarbazole; or a conventional amine derivative having an aromatic condensation ring, such as N,N′-bis(3-methylphenyl)-N,N′-diphenyl-[1,1 -biphenyl]-4,4′-diamine (TPD), or N,N′-di(naphtalene-1-yl)-N,N′-diphenyl benzidine (α-NPD).

The thickness of the HTL may be in a range of about 50 Å to 1000 Å, specifically 100 Å to 600 Å. If the thickness of the HTL is less than 50 Å, hole transporting properties may be degraded. On the other hand, if the thickness of the HTL is greater than 1000 Å, the driving voltage of the organic light emitting device may be increased.

Then, an emission layer (EML) may be formed on the HTL by using a vacuum deposition method, a spin coating method, a casting method, or an LB deposition method. When the EML is formed by using a vacuum deposition method or a spin coating method, the deposition and coating conditions may differ according to the type of an EML forming material, and can be very similar to the deposition and coating conditions used to form the HIL.

The EML may be formed using the indeno indene-based compound represented by Formula 1 according to an embodiment of the present invention. According to an embodiment of the present invention, the EML may include only the indeno indene-based compound represented by Formula 1. According to another embodiment of the present invention, the EML may include a known host or dopant material as well as the indeno indene-based compound represented by Formula 1, Examples of the host material include (8-quinolinorato)aluminum (Alq₃), 4,4′-N,N′-dicarbazole-biphenyl (CBP), and poly(n-vinylcarbazole) (PVK).

The dopant material can be a fluorescent dopant or a phosphorescent dopant. Examples of the fluorescent dopant include IDE102 and IDE105 (manufactured by Idemitsu Kosan Co., Ltd.) and C545T (manufactured by Hayashibara Co., Ltd.). The phosphorescent dopant can be categorized into a red phosphorescent dopant, a green phosphorescent dopant, and a blue phosphorescent dopant. The red phosphorescent dopant may be PtOEP, or RD 61 (manufactured by UDC Co.). The green phosphorescent dopant may be Ir(PPy)3 where PPy denotes 2-phenylpyridine. The blue phosphorescent dopant may be F2Irpic. For example, the dopant material may be a compound represented by Formula 8:

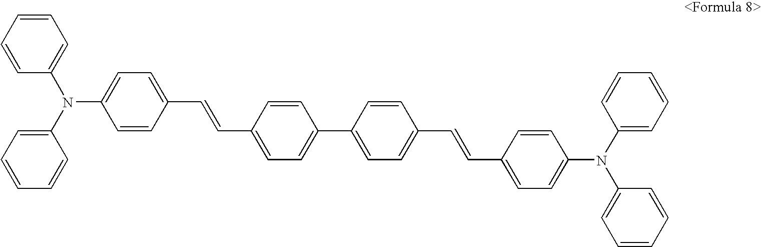

The doping concentration may not be limited and may be in a range of 0.01-15 parts by weight based on 100 parts by weight of the host.

The thickness of the EML may be in a range of about 100 Å to 1000 Å, specifically 200 Å to 600 Å, but is not limited thereto. If the thickness of the EML is less than 100 Å, emission properties of the EML may be degraded. On the other hand, if the thickness of the EML is greater than 1000 Å, the driving voltage of the organic light emitting device may be increased.

However, when the EML is formed using a phosphorescent dopant, triplet excitons or holes can diffuse toward an electron transport layer. The diffusion of triplet excitons or holes can be prevented by forming a hole blocking layer (HBL) on the EML using a vacuum deposition method, a spin coating method, a casting method, or a LB deposition method. When the HBL is formed by using a vacuum deposition method or a spin coating method, the deposition and coating conditions may differ according to the type of the HBL forming material, and can be very similar to the deposition and coating conditions used to form the HIL. The HBL forming material may be, for example, an oxadiazole derivative, a triazole derivative, a phenanthroline derivative, a hole blocking material disclosed in JP 11-329734(A1) which is incorporated herein by reference, or bathocuproine (BCP).

The thickness of the HBL may be in a range of about 50 Å to 1,000 Å, specifically 100 Å to 300 Å. If the thickness of the HBL is less than 50 Å, hole blocking properties may be degraded. On the other hand, if the thickness of the HBL is greater than 1,000 Å, the driving voltage of the organic light emitting device may be increased.

Then, an electron transport layer (ETL) can be formed on the HBL by using a vacuum deposition method, a spin coating method, or a casting method. When the ETL is formed by using a vacuum depositing method or a spin coating method, the deposition and coating conditions may differ according to the type of the ETL forming material and can be very similar to the deposition and coating conditions used to form the HIL. The ETL forming material may be any known material that stably transports electrons injected from an electron injection electrode, that is, a cathode. For example, the ETL forming material may be a quinoline derivative, such as tris(8-quinolinorate)aluminum Alq₃, 3-phenyl-4-(1′-naphthyl)-5-phenyl-1,2,4-triazole (TAZ), or aluminum(III)bis(2-methyl-8-quinolinato)4-phenylphenolate (Balq).

The thickness of the ETL may be in a range of about 100 Å to 1000 Å, specifically 200 Å to 500 Å. If the thickness of the ETL is less than 100 Å, electron transporting properties may be degraded. On the other hand, if the thickness of the ETL is greater than 1000 Å, the driving voltage of the organic light emitting device may be increased.

In addition, an electron injection layer (EIL) forming material that allows electrons to be easily injected from the cathode can be deposited on the ETL to form an EIL, and is not limited.

The EIL forming material may be any known EIL forming material, such as LiF, NaCl, CsF, Li₂O, or BaO. The deposition conditions of the ETL may differ according to the type of the EIL forming material and can be very similar to the deposition and coating conditions used to form the HIL.

The thickness of the EIL may be in a range of about 1 Å to 100 Å, specifically 5 Å to 50 Å. If the thickness of the EIL is less than 1 Å, electron injection properties may be degraded. On the other hand, if the thickness of the EIL is greater than 100 Å, the driving voltage of the organic light emitting device may be increased.

Finally, a second electrode can be formed on the EIL by applying a second electrode forming material to the EIL using a vacuum deposition method or a sputtering method. The second electrode may function as a cathode. The second electrode forming material may be a metal, an alloy, an electroconductive compound, or a mixture thereof, each having a low work function. For example, the second electrode forming metal may be Li, Mg, Al, Al—Li, Ca, Mg—In, or Mg—Ag. If the organic light emitting device is a top-emission type light emitting device, the second electrode can be a transmissive cathode formed of ITO or IZO.

An organic light emitting device according to the present invention can also include, in addition to the structure of first electrode/HIL/HTL/EML/HBL/ETL/EIL/second electrode illustrated in FIG. 1C, other structures, for example, the structure of the organic light emitting device illustrated in FIG. 1A, which will be described in detail in Examples 1 and 2.

The present invention will be now described in further detail with reference to the following examples in which Compounds 2-11 according to the present invention are respectively represented by Formulae 2-11. These examples are for illustrative purposes only and are not intended to limit the scope of the present invention.

### SYNTHESIS EXAMPLE 1

Compound 2 was Prepared.

Glass transition temperature (Tg), melting point (Tm), thermal degradation temperature (Td) and highest occupied molecular orbit (HOMO) of Compound 2 were measured. The results are shown in Table 1.

TABLE 1

| Compound No. | Tg (° C.) | Tm (° C.) | Td (° C.) | HOMO |
|---|---|---|---|---|
| 2 | 128 | 273 | 418 | 5.87 |

Evaluation 1—Thermal Stability

The thermal stability of Compound 2 was measured based on Tg and Tm. Meanwhile, Tg and Tm of Comparative Compound A having the following structure (hereinafter referred to as “Compound A) were measured:

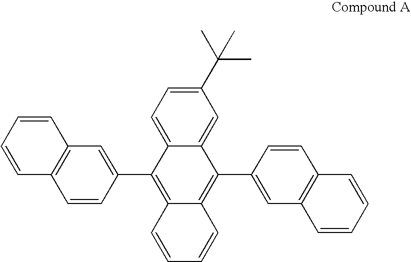

Tg and Tm were measured by performing a thermal analysis using thermo gravimetric analysis (TGA) and differential scanning calorimetry (DSC). The results are shown in Table 2:

TABLE 2

| Compound No. | Tg (° C.) | Tm (° C.) |
|---|---|---|
| A | 129 | 291 |

Therefore, it can be seen that a compound according to the present invention has a thermal stability suitable for use in an organic light emitting device.

Evaluation 2: Emission Properties of Compound 2

Emission properties of Compound 2 were identified by measuring UV absorption spectrum and photoluminescence (PL) spectrum. First, Compound 2 was diluted with toluene such that the concentration of the resultant solution was 0.2 mM, and then UV absorption spectrum of Compound 2 was measured with a Shimadzu UV-350 spectrometer. Then, the same experiment was performed on Compound A. Meanwhile, Compound 2 was diluted with toluene such that the concentration of the resultant solution was 10 mM, and then a PL spectrum of Compound 2 was measured with an ISC PC1 spectrofluorometer equipped with a Xenon lamp. Then, the same experiment was performed on Compound A. The results are shown in Table 3.

TABLE 3

| | Absorption | Maximum PL |
|---|---|---|
| Compound | wavelength | wavelength |
| No. | (nm) | (nm) |
| A | 397, 378 | 432 |

Therefore, it can be seen that a compound according to the present invention has emission properties suitable for use in an organic light emitting device.

Evaluation 3: Emission Properties of Compound 2 (in Film State)

Compound 2 was prepared in the form of a film, and the absorption spectrum, PL spectrum, PCS properties, and quantum efficiency of the film were measured.

First, a quartz substrate was washed with chloroform and pure water. Then, Compound 2 was spin coated on the quartz substrate and then heat-treated at 110° C. for 2 hours, thereby forming a film having a thickness of 1,000 Å. The obtained film will be referred to as Film 2. The absorption spectrum, PL spectrum, and quantum efficiency of Film 2 were measured with the same equipment used in Evaluation 2.

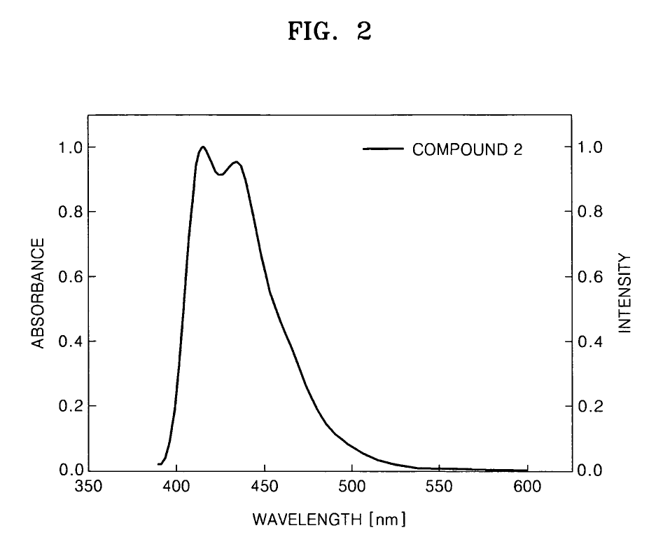

FIG. 2 illustrates absorption and PL spectra of Film 2.

Referring to FIG. 2, when a compound according to an embodiment of the present invention is prepared in the form of a film, the film has appropriate absorption spectrum, PL spectrum, and quantum efficiency suitable for use in an organic light emitting device.

### EXAMPLE 1

An organic light emitting device having the structure below was manufactured, wherein an emission layer was formed using a compound represented by Formula 8 acting as a dopant (hereinafter referred to as ‘dopant E605’) and Compound 2 acting as a host: ITO/PEDOT(500Å)/Compound 2_dopant E605(480Å)/Alq₃(200Å)/LiF(10Å)/Al(2000Å).

A 15Ω/cm² (1000Å) ITO glass substrate was cut to a size of 50 mm×50 mm×0.7 mm, and then sonicated with acetone isopropyl alcohol and pure water each for 15 minutes and washed with UV and ozone for 30 minutes, thereby forming an anode. Then, PEDOT-PSS (Al4083) manufactured by Bayer Co. was coated on the anode and then heat-treated at 110° C. for 5 minutes in air and heat-treated at 200° C. for 5 minutes in a nitrogen atmosphere, thereby forming a hole injection layer having a thickness of 500 Å. Then, a mixture including 1 g of Compound 2 acting as a host and 0.05 g of E605 was spin coated on the hole injection layer and then heat-treated at 110° C. for 30 minutes, thereby forming an emission layer having a thickness of 480 Å, wherein the weight of E605 was 5 parts by weight based on 100 parts by weight of Compound 2. Then, an Alq₃ compound was vacuum deposited on the emission layer to form an electron transport layer having a thickness of 200 Å. Then, LiF was vacuum deposited on the electron transport layer to form an electron injection layer having a thickness of 10 Å, and Al was vacuum deposited on the electron injection layer to form a cathode having a thickness of 2000 Å, thereby completing the manufacture of an organic light emitting device having the structure illustrated in FIG. 1B. The obtained organic light emitting device will be referred to as Sample 1.

### EXAMPLE 2

An organic light emitting device having the structure below was manufactured in the same manner as in Example 1, except that the emission layer was formed using only Compound 2: ITO/PEDOT(500Å)/Compound 2(480Å)/Alq₃(200Å)/LiF(10 Å)/Al(2000Å). The obtained organic light emitting device will be referred to as Sample 2.

### COMPARITIVE EXAMPLE A

An organic light emitting device having the structure below was manufactured in the same manner as in Example 1, except that Compound A was used instead of Compound 2 as a host:ITO/PEDOT(500Å)/Compound A_dopant E605(480Å)/Alq₃(200Å)/LiF(10Å)/Al(2000Å). The obtained organic light emitting device will be referred to as Sample A.

TABLE 4

| | | CIE color | |
|---|---|---|---|
| Sample | Turn on | coordinate | Efficiency |
| No. | voltage (V) | (~100 cd/m²) | (cd/A) |
| A | 3.4 | 0.15, 0.27 | 4.16 |
| 1 | 3.1 | 0.14, 0.23 | 5.21 |
| 2 | 3.3 | 0.14, 0.25 | 4.92 |

From Table 4, it can be seen that Sample 1 according to the present invention has excellent electrical properties.

As described above, the indeno indene-based compound according to an embodiment of the present invention has having excellent electrical properties, thermal stability and photochemical stability, and is capable of improving driving voltage properties, efficiency, and color purity when used in an organic light emitting device.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An indeno indene-based compound represented by Formula 1: 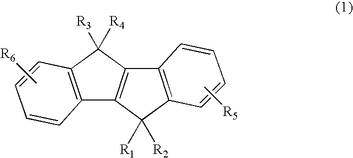
where R₁, R₂, R₃, R₄, R₅ and R₆ are each independently, hydrogen, a substituted or unsubstituted C₁-C₄₀ alkyl group, a substituted or unsubstituted C₁-C₄₀ alkoxy group, a substituted or unsubstituted C₆-C₅₀ aryl group, a substituted or unsubstituted C₂-C₅₀ heteroaryl group, a substituted or unsubstituted C₅-C₄₀ cycloalkyl group, a substituted or unsubstituted C₅-C₄₀ hetero cycloalkyl group, —N(Q₁)(Q₂) or —Si(Q₃)(Q₄)(Q₅) where Q₁, Q₂, Q₃, Q₄ and Q₅ are each independently, hydrogen, a substituted or unsubstituted C₆-C₅₀ aryl group, a substituted or unsubstituted C₂-C₅₀ heteroaryl group, a substituted or unsubstituted C₅-C₄₀ cycloalkyl group or a substituted or unsubstituted C₅-C₄₀ hetero cycloalkyl group.

2. The indeno indene-based compound of claim 1, wherein the substituted C₁-C₄₀ alkyl group, the substituted C₁-C₄₀ alkoxy group, the substituted C₆-C₅₀ aryl group, the substituted C₂-C₅₀ heteroaryl group, the substituted C₅-C₄₀ cycloalkyl group, and the substituted C₅-C₄₀ hetero cycloalkyl group have at least one substituent independently selected from the group consisting of —F, —Cl, —Br, —CN, —NO₂, —OH, a C₁-C₅₀ alkyl group that is unsubstituted or substituted with —F, —Cl, —Br, —CN, —NO₂ or —OH, a C₁-C₅₀ alkoxy group that is unsubstituted or substituted with —F, —Cl, —Br, —CN, —NO₂ or —OH, a C₆-C₅₀ aryl group that is unsubstituted or substituted with a C₁-C₅₀ alkyl group, a C₁-C₅₀ alkoxy group, —F, —Cl, —Br, —CN, —NO₂ or —OH, a C₂-C₅₀ heteroaryl group that is unsubstituted or substituted with a C₁-C₅₀ alkyl group, a C₁-C₅₀ alkoxy group, —F, —Cl, —Br, —CN, —NO₂ or —OH, a C₅-C₅₀ cycloalkyl group that is unsubstituted or substituted with a C₁-C₅₀ alkyl group, a C₁-C₅₀ alkoxy group, —F, —Cl, —Br, —CN, —NO₂ or —OH, a C₅-C₅₀ heterocycloalkyl group that is unsubstituted or substituted with a C₁-C₅₀ alkyl group, a C₁-C₅₀ alkoxy group, —F, —Cl, —Br, —CN, —NO₂ or —OH; and a group represented by —N(Q₁₁)(Q₁₂) where Q₁₁ and Q₁₂ are each independently hydrogen, a C₁-C₅₀ alkyl group, or a C₆-C₅₀ aryl group substituted with a C₁-C₅₀ alkyl group.

3. The indeno indene-based compound of claim 1, wherein R₁, R₂, R₃, R₄, R₅ and R₆ are each independently hydrogen, a C₁-C₄₀ alkyl group, a C₁-C₄₀ alkoxy group, a phenyl group, a biphenyl group, a pentalenyl group, an indenyl group, a naphthyl group, a biphenylenyl group, an anthracenyl group, an azurenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolinyl group, a piperidinyl group, a piperazinyl group, a morpholinyl group, a di(C₆-C₅₀ aryl)amino group, a tri(C₆-C50aryl)silyl group, or derivatives thereof.

4. The indeno indene-based compound of claim 1, wherein R₁, R₂, R₃, R₄, R₅ and R₆ are each independently selected from the group consisting of aryl groups having the following structures: 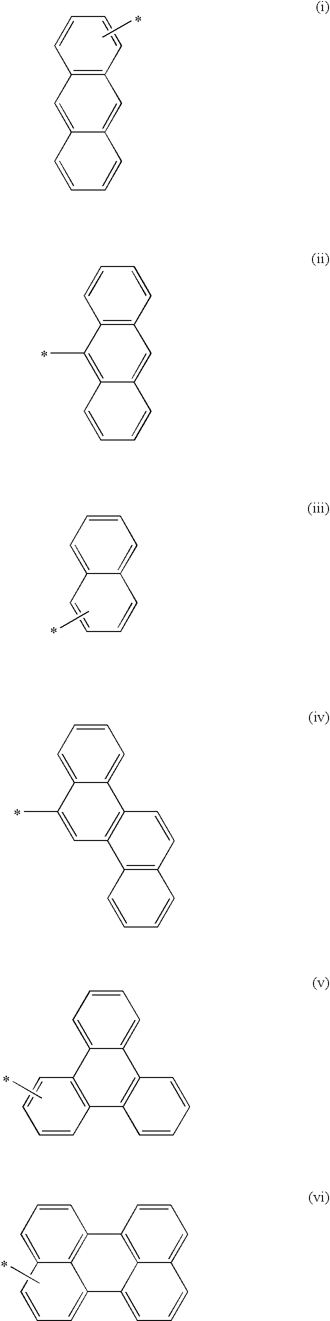 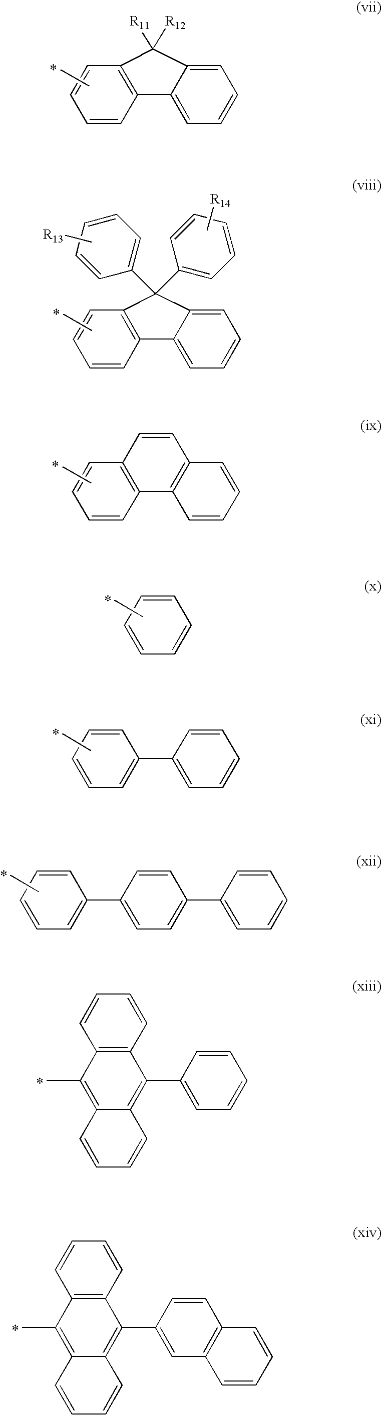 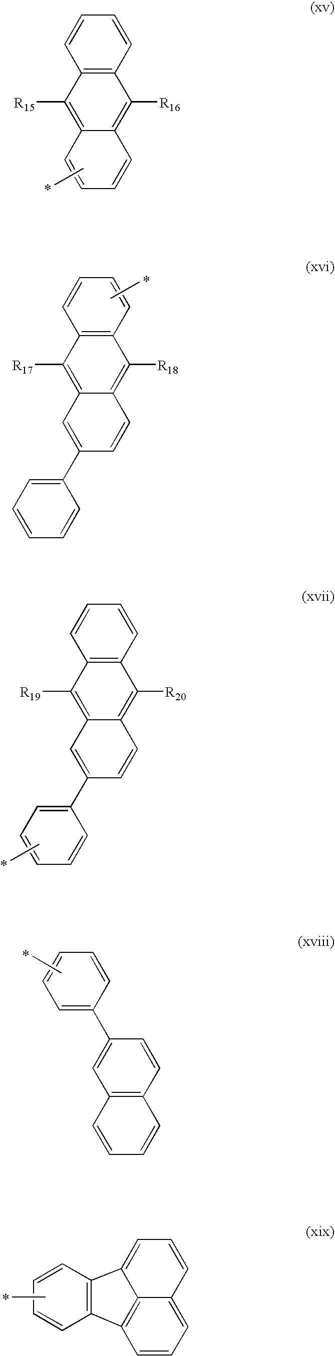 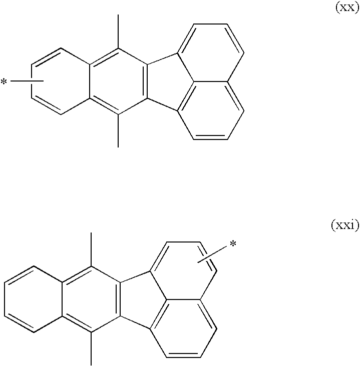
where R₁₁, R₁₂, R₁₃, R₁₄, R₁₅, R₁₆, R₁₇, R₁₈, R₁₉ and R₂₀ are each independently, hydrogen, a substituted or unsubstituted C₁-C₄₀ alkyl group, a substituted or unsubstituted C₆-C₅₀ aryl group, a substituted or unsubstituted C₂-C₅₀ heteroaryl group, a substituted or unsubstituted C₅-C₄₀ cycloalkyl group or a substituted or unsubstituted C₅-C₄₀ hetero cycloalkyl group; and
an asterisk (*) indicates a binding site.

5. The indeno indene-based compound of claim 1, wherein at least one of R₁ and R₂ and at least one of R₃ and R₄ are each independently selected from the group consisting of a methyl group and functional groups represented by Formulae iii, x, xviii and xxii:

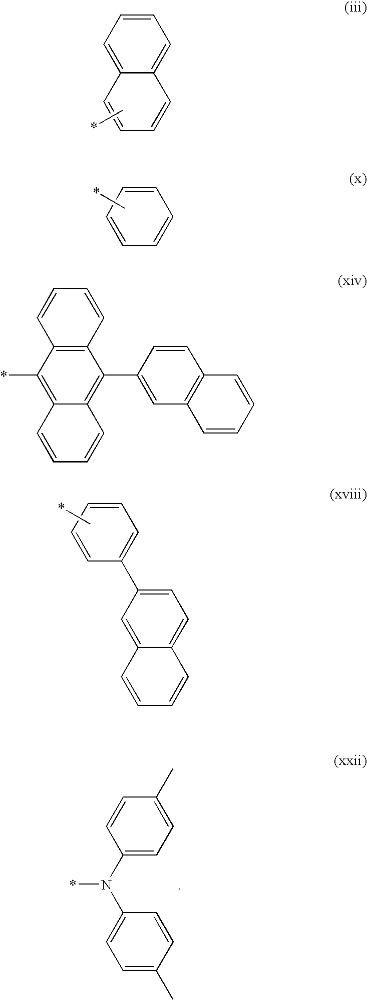

6. The indeno indene-based compound of claim 1, wherein the compound represented by Formula 1 is selected from the group consisting of compounds represented by Formulae Formula 2 through 7:

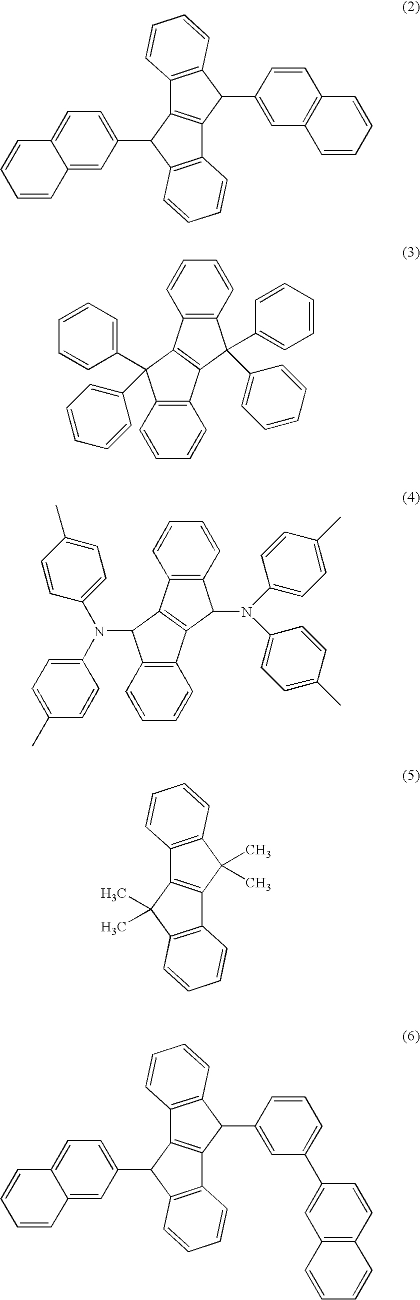

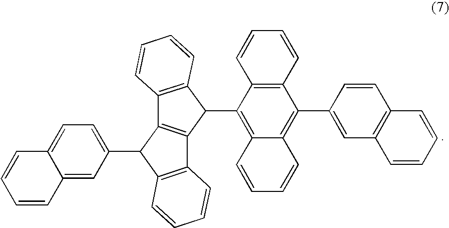

7. An organic light emitting device comprising an organic layer comprised of the indeno indene-based compound of claim 1.

8. An organic light emitting device comprising:
a first electrode;
a second electrode; and
at least one organic layer between the first electrode and the second electrode,said at least one organic layer comprising an organic layer comprised of an indeno indene-based compound represented by Formula 1: 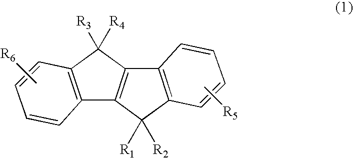
where R₁, R₂, R₃, R₄, R₅ and R₆ are each independently, hydrogen, a substituted or unsubstituted C₁-C₄₀ alkyl group, a substituted or unsubstituted C₁-C₄₀ alkoxy group, a substituted or unsubstituted C₆-C₅₀ aryl group, a substituted or unsubstituted C₂-C₅₀ heteroaryl group, a substituted or unsubstituted C₅-C₄₀ cycloalkyl group, a substituted or unsubstituted C₅-C₄₀ hetero cycloalkyl group, —N(Q₁)(Q₂) or —Si(Q₃)(Q₄)(Q₅) where Q₁, Q₂, Q₃, Q₄ and Q₅ are each independently, hydrogen, a substituted or unsubstituted C₆-C₅₀ aryl group, a substituted or unsubstituted C₂-C₅₀ heteroaryl group, a substituted or unsubstituted C₅-C₄₀ cycloalkyl group or a substituted or unsubstituted C₅-C₄₀ hetero cycloalkyl group.

9. The organic light emitting device of claim 8, wherein the organic layer comprised of the indeno indene-based compound is selected from the group consisting of a hole injection layer, a hole transport layer, an electron blocking layer, an emission layer, a hole blocking layer, an electron transport layer and an electron injection layer.

10. The organic light emitting device of claim 8, further comprising: between the first electrode and the second electrode, at least one layer selected from the group consisting of a hole injection layer, a hole transport layer, an electron blocking layer, a hole blocking layer, an electron transport layer, and an electron injection layer.

11. The organic light emitting device of claim 8, wherein R₁, R₂, R₃, R₄, R₅ and R₆ are each independently hydrogen, a C₁-C₄₀ alkyl group, a C₁-C₄₀ alkoxy group, a phenyl group, a biphenyl group, a pentalenyl group, an indenyl group, a naphthyl group, a biphenylenyl group, an anthracenyl group, an azurenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolinyl group, a piperidinyl group, a piperazinyl group, a morpholinyl group, a di(C₆-C₅₀ aryl)amino group, a tri(C₆-C₅₀aryl)silyl group, or derivatives thereof.

12. The organic light emitting device of claim 8, wherein at least one of R₁, R₂, R₃, R₄, R₅ and R₆ is selected from the group consisting of aryl groups having the following structures: 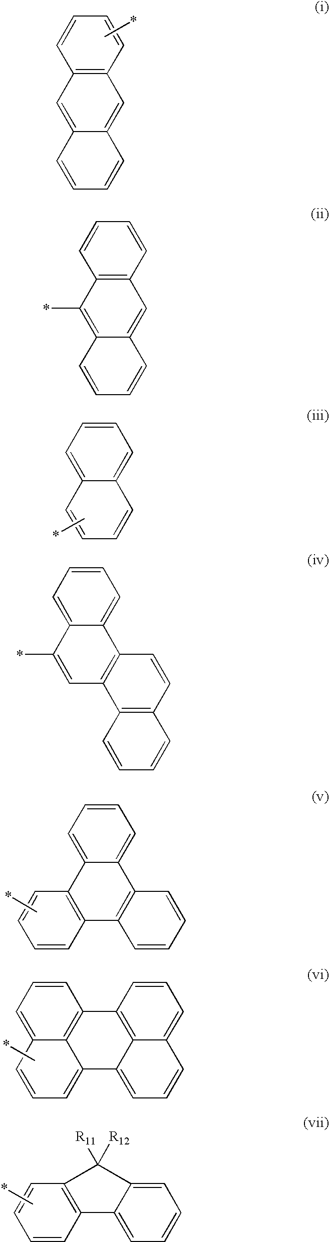 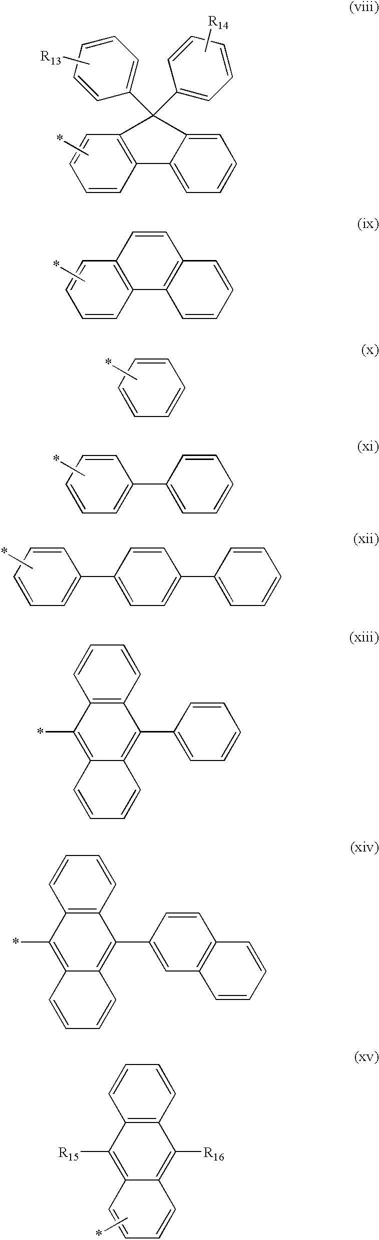 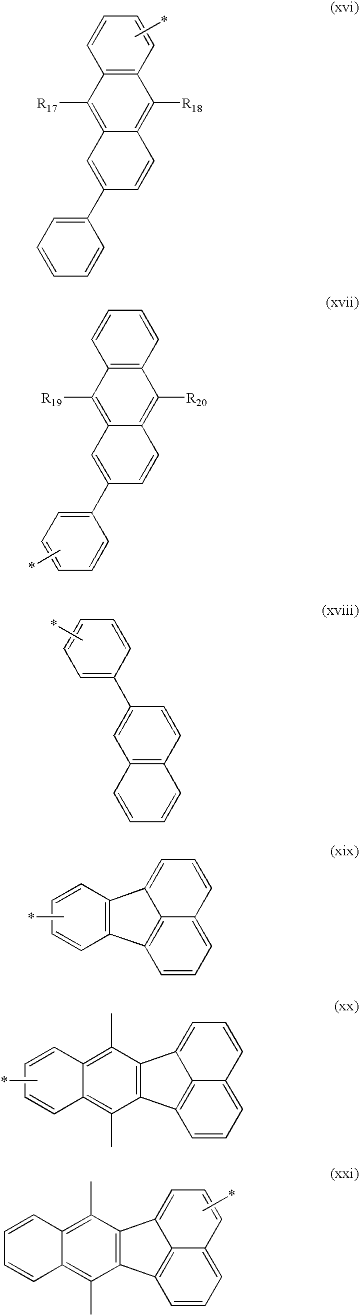
where R₁₁, R₁₂, R₁₃, R₁₄, R₁₅, R₁₆, R₁₇, R₁₈, R₁₉ and R₂₀ are each independently, hydrogen, a substituted or unsubstituted C₁-C₄₀ alkyl group, a substituted or unsubstituted C₆-C₅₀ aryl group, a substituted or unsubstituted C₂-C₅₀ heteroaryl group, a substituted or unsubstituted C₅-C₄₀ cycloalkyl group or a substituted or unsubstituted C₅-C₄₀ hetero cycloalkyl group.

13. The organic light emitting device of claim 8, wherein at least one of R₁ and R₂, and at least one of R₃ and R₄ are each independently selected from the group consisting of a methyl group and functional groups represented by Formulae iii, x, xviii and xxii:

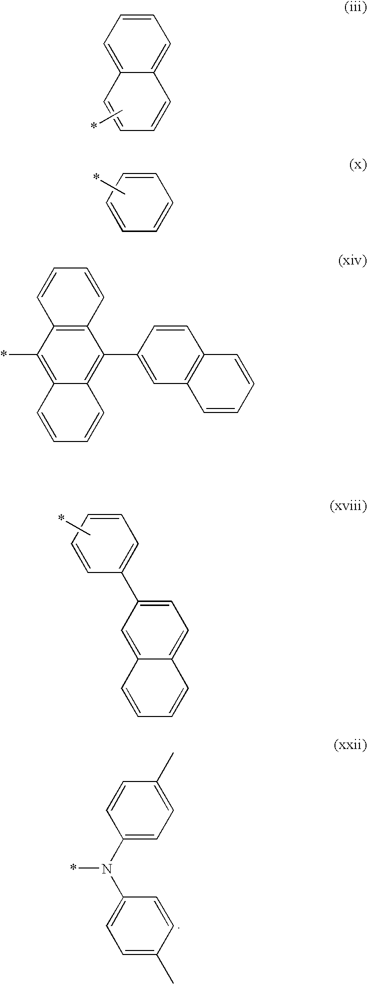

14. The organic light emitting device of claim 8, wherein the compound represented by Formula 1 is selected from the group consisting of compounds represented by Formulae Formula 2 through 4:

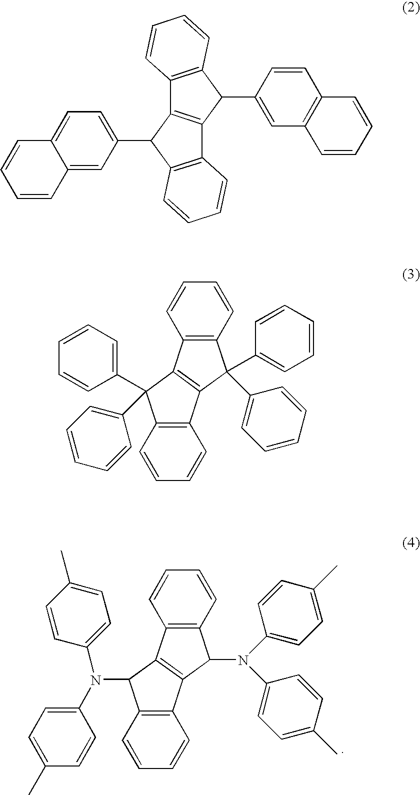

15. The organic light emitting device of claim 8, wherein the compound represented by Formula 1 is selected from the group consisting of compounds represented by Formulae Formula 5 through 7:

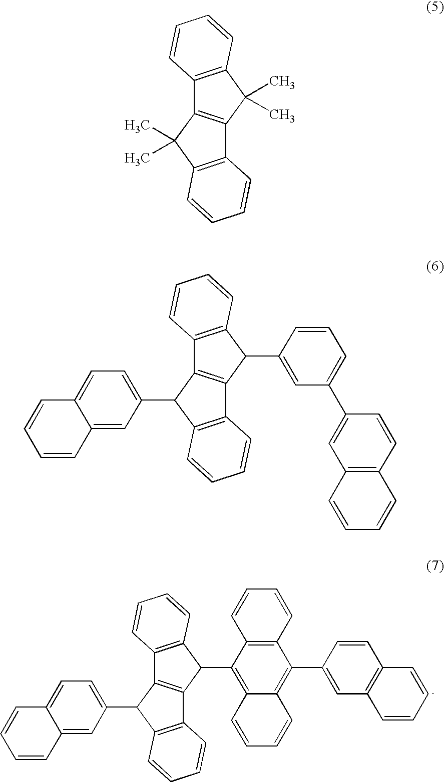

16. A method of manufacturing an organic light emitting device, the method comprising:
forming a first electrode on a substrate;
forming at least one organic layer comprising an organic layer comprised of an indeno indene-based compound on the first electrode, the indeno indene-based compound represented by Formula 1: 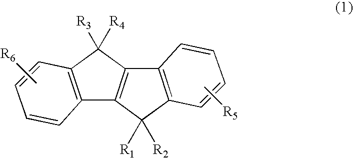
where R₁, R₂, R₃, R₄, R₅ and R₆ are each independently, hydrogen, a substituted or unsubstituted C₁-C₄₀ alkyl group, a substituted or unsubstituted C₁-C₄₀ alkoxy group, a substituted or unsubstituted C₆-C₅₀ aryl group, a substituted or unsubstituted C₂-C₅₀ heteroaryl group, a substituted or unsubstituted C₅-C₄₀ cycloalkyl group, a substituted or unsubstituted C₅-C₄₀ hetero cycloalkyl group, —N(Q₁)(Q₂) or —Si(Q₃)(Q₄)(Q₅) where Q₁, Q₂, Q₃, Q₄ and Q₅ are each independently, hydrogen, a substituted or unsubstituted C₆-C₅₀ aryl group, a substituted or unsubstituted C₂-C₅₀ heteroaryl group, a substituted or unsubstituted C₅-C₄₀ cycloalkyl group or a substituted or unsubstituted C₅-C₄₀ hetero cycloalkyl group; and
forming a second electrode on the at least one organic layer.

17. The method of claim 16, wherein the organic layer comprised of the indeno indene-based compound is selected from the group consisting of a hole injection layer, a hole transport layer, an electron injection layer, an electron transport layer, and an emission layer.

18. The method of claim 16, wherein the organic layer comprised of the indeno indene-based compound is formed using any one method selected from the group consisting of a deposition method, a spin coating method, a casting method, an inkjet printing method, and a microcontact printing method.

19. The method of claim 16, wherein the formation of said at least one organic layer comprises at least one process selected from the group consisting of a hole injection layer forming process, a hole transport layer forming process, an electron blocking layer forming process, a hole blocking layer forming process, an electron transport layer forming process, and an electron injection layer forming process.

20. The organic light emitting device manufactured by the method of claim 16.
